(19) United States
(12) Patent Application Publication
(10) Pub. No.: US 2025/0122308 A1
(43) Pub. Date: Apr. 17, 2025

(54) TARGETING MUTANT KRAS WITH A MUTATION SPECIFIC IGA

(71) Applicant: H. LEE MOFFITT CANCER CENTER AND RESEARCH INSTITUTE, INC., Tampa, FL (US)

(72) Inventors: Jose CONEJO-GARCIA, Tampa, FL (US); Subir BISWAS, Tampa, FL (US)

(21) Appl. No.: 18/692,500

(22) PCT Filed: Sep. 16, 2022
(86) PCT No.: PCT/US2022/076578

§ 371 Date: Mar. 15, 2024

Related U.S. Application Data
(60) Provisional application No. 63/245,269, filed on Sep. 17, 2021.
(60) Provisional application No. 63/288,182, filed on Dec. 10, 2021.
(60) Provisional application No. 63/353,339, filed on Jun. 17, 2022.
(60) Provisional application No. 63/396,945, filed on Aug. 10, 2022.

(51) Int. Cl. C07K 16/40; A61K 39/00; A61P 35/00

(52) U.S. Cl. CPC C07K 16/40; A61P 35/00; C12Y 306/05002; A61K 2039/505; C07K 2317/33

(57) ABSTRACT

Disclosed herein are dimeric or pentameric antigen binding molecules and methods for making said dimeric or pentameric antigen binding molecules, wherein the isotype of the immunoglobulin monomers is IgA, 1gG4, or IgM. Also disclosed herein are methods of using dimeric antigen binding molecules to treat cancer associated with expression of an oncogene, gene overexpression, or a gene fusion.

This application claims the benefit of U.S. Provisional Application No. 63/396,945, filed on Aug. 10, 2022; U.S. Provisional Application No. 63/353,339, filed on Jun. 17, 2022; U.S. Provisional Application No. 63/288,182, filed on Dec. 10, 2021; and U.S. Provisional Application No. 63/245,269, filed on Sep. 17, 2021, applications which are incorporated herein by reference in their entireties.

This invention was made with government support under Grant No. CA232758 awarded by the National Institutes of Health. The government has certain rights in the invention.

### I. BACKGROUND

Despite the investment of billions of dollars and years of research successful cancer treatments for many cancers is still an elusive goal. What are needed are new therapeutics that can attack and eliminate a cancer.

### II. SUMMARY

Disclosed are methods and compositions related to dimeric antigen binding molecules.

In one aspect, disclosed herein are dimeric IgA, dimeric IgG4, or pentameric IgM antigen binding molecule; wherein the antigen binding molecule specifically binds ABL1, ABL2, ASCL1, AKT1, AKT2, ALK, APC, AR, ARID1A, ARID2, ATM, BCLAF1, BRAF, BRCA1, BRCA2, CCND3, CTNNB1, CREBBP, DNMT3A, EGFR, EP300, ERBB2, ERBB3, ESR1, EZH2, FAT1, FAT3, FAT4, FBXO11, FBXW7, FGFR1, FLT3, FOXA1, GNA11, GNAQ, GNAS, GTF2I, H3F3A, HER2/NEU, HRAS, IDH1, IDH2, JAK2, KCNJ4, KDM6A, KIT, KMT2C, KMT2D, KRAS, LRP1B, MET, MTOR, MYC, MYCN, NF1, NOTCH1, NRAS, NSD1, NSD2, NTRK3, OBSCN, PCBP1, PIK3CA, PIK3R1, PIGR, PLCG1, PTCH1, PTEN, PTPN6, PTPN11, PTPN13, RAC1, RB1, RET, ROS1, RHOA, RYR2, SET2D, SF3B1, SMAD2, SMAD4, SMARCA4, SRC, SRSF2, TP53, TRRAP, TTN, U2AF1L4, VHL, or CDKN2A or a cancer associated gene overexpression or gene fusion (such as, for example, ALK fusions including, but not limited to EML4-ALK, KIF5B-ALK, HIP1-ALK, KLC1-ALK, DCTN1-ALK, PRKAR1A-ALK, STRN-ALK, CLTC-ALK, MPRIP-ALK, NPM1-ALK, TNS1-ALK, ACTG2-ALK, IGFBP5-ALK, SEC31A-ALK, TPM3-ALK, AITC-ALK, TPM4-ALK; ROS1 fusions including, but not limited to, CD74 ROS1, SLC34A2-ROS1, SDC4-ROS1, EZR-ROS1, LR1G3-ROS1, SDC4-ROS1, TPM3-ROS1, LIMA1-ROS1, MSN-ROS1, WNK1-ROS1, RBPMS-ROS1; or RET fusions including, but not limited to, KIF5B-RET, CCDC6-RET, and TRIM33-RET); and wherein antigen binding molecule comprising two immunoglobulin monomers comprising a heavy chain, a light chain, and a constant region; wherein the constant regions of the monomers are joined by a J chain. For example, disclosed herein are dimeric or pentameric anti-KRAS^{G12D}-specific, anti-KRAS^{G12C}-specific, anti-KRAS^{G12V}-specific, anti-AKT1^{E17K}-specific, anti-AKT2^{D324G}-specific, anti-AKT2^{D324H}-specific, anti-IDH1^{R132H}-specific, anti-IDH1^{R132C}-specific, anti-IDH1^{R132L}-specific, anti-ATM^{R337C}-specific, anti-ATM^{R337H}-specific, anti-PI3K^{E542K}-specific, anti-PI3K^{E545K}-specific, anti-PI3K^{H1047R}-specific, anti-BRAF^{V600G}-specific, anti-BRAF^{V600E}-specific, anti-SRC^{D407H}-specfic, anti-p53^{R273C}-specific, and/or anti-p53^{R273H}-specific antigen binding molecules comprising two immunoglobulin monomers (such as, for example two IgA or two IgG4) or 5 immunoglobulin monomer (such as, for example, five IgM monomers) comprising a heavy chain, a light chain, and a constant region; wherein the constant regions of the monomers are joined by a J chain.

Also disclosed herein are dimeric or pentameric anti-KRAS^{G12D}-specific antigen binding molecules of any preceding aspect (such as, for example, dimeric IgA, dimeric IgG4, or pentameric IgM) wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8; SEQ ID NO: 43, SEQ ID NO: 44, and SEQ ID NO: 45; or SEQ ID NO: 46, SEQ ID NO: 47, and SEQ ID NO: 48, including, but not limited to SEQ ID NO: 5 or SEQ ID NO: 49. In some aspects, the heavy chain variable domain of the dimeric anti-KRAS^{G12D}-specific antigen binding molecules comprises SEQ ID NO: 1, SEQ ID NO: 32, and SEQ ID NO: 34.

In some aspects, disclosed herein are dimeric or pentameric anti-KRAS^{G12D}-specific antigen binding molecules of any preceding aspect (such as, for example, dimeric IgA, dimeric IgG4, or pentameric IgM), wherein the constant region in frame with the heavy chain variable region comprises the sequence as set forth in SEQ ID NO: 2.

Also disclosed herein are dimeric or pentameric anti-KRAS^{G12D}-specific antigen binding molecules of any preceding aspect (such as, for example, dimeric IgA, dimeric IgG4, or pentameric IgM), wherein the light chain variable domain comprises a complementarity determining region (CDR) 1 (CDR1), CDR2, and CDR3 as set forth in SEQ ID NO: 10, SEQ ID NO: 11, and SEQ ID NO: 12 or SEQ ID NO: 50, SEQ ID NO: 51, and SEQ ID NO: 52, including, but not limited to SEQ ID NO: 9 and SEQ ID NO: 53. In some aspects, the light chain variable domain of the dimeric anti-KRAS^{G12D}-specific antigen binding molecules comprises SEQ ID NO: 3 and SEQ ID NO: 36.

In some aspects, disclosed herein are dimeric or pentameric anti-KRAS^{G12D}-specific antigen binding molecules of any preceding aspect (such as, for example, dimeric IgA, dimeric IgG4, or pentameric IgM), wherein the constant region in frame with the light chain variable region comprises the sequence as set forth in SEQ ID NO: 4.

Also disclosed herein are dimeric or pentameric anti-IDH1^{R132H}-specific antigen binding molecule of any preceding aspect (such as, for example, dimeric IgA, dimeric IgG4, or pentameric IgM), wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8 or SEQ ID NO: 16, SEQ ID NO: 17, and SEQ ID NO: 18. In some aspects, the variable heavy chain comprises the amino acid sequence as set forth in SEQ ID NO: 15, SEQ ID NO: 38, SEQ ID NO: 40, or SEQ ID NO: 54.

In some aspects, disclosed herein are dimeric or pentameric anti-IDH1^{R132H}-specific antigen binding molecule of any preceding aspect (such as, for example, dimeric IgA, dimeric IgG4, or pentameric IgM), wherein the light chain variable domain comprises a complementarity determining region (CDR) 1 (CDR1), CDR2, and CDR3 as set forth in SEQ ID NO: 10, SEQ ID NO: 11, and SEQ ID NO: 12 or SEQ ID NO: 20, SEQ ID NO: 21, and SEQ ID NO: 22. In some aspects, the variable light chain comprises the amino acid sequence as set forth in SEQ ID NO: 19, SEQ ID NO: 42, or SEQ ID NO: 55.

Also disclosed herein are dimeric or pentameric anti-anti-ASCL1-specific antigen binding molecules of any preceding aspect (such as, for example, dimeric IgA, dimeric IgG4, or pentameric IgM) wherein the heavy chain variable domain comprises a complementarity determining region (CDR) 1 (CDR1), CDR2, and CDR3 as set forth in SEQ ID NO: 62, SEQ ID NO: 63, and SEQ ID NO: 64. In some aspects, the heavy chain variable domain of the dimeric anti-ASCL1 specific binding molecule comprises SEQ ID NO: 60 or SEQ ID NO: 61.

In some aspects disclosed herein are dimeric or pentameric anti-anti-ASCL1-specific antigen binding molecules of any preceding aspect (such as, for example, dimeric IgA, dimeric IgG4, or pentameric IgM) wherein the light chain variable domain comprises a complementarity determining region (CDR) 1 (CDR1), CDR2, and CDR3 as set forth in SEQ ID NO: 67, SEQ ID NO: 68, and SEQ ID NO: 69. In some aspects, the light chain variable domain of the dimeric anti-ASCL1 specific binding molecule comprises SEQ ID NO: 65 or SEQ ID NO: 66.

Also disclosed herein are dimeric or pentameric antigen binding molecules of any preceding aspect (such as, for example, dimeric IgA, dimeric IgG4, or pentameric IgM), wherein the isotype of the immunoglobulin monomers is IgA, IgG4, or IgM.

Also disclosed herein are methods of treating, inhibiting, decreasing, reducing, ameliorating, and/or preventing a cancer and/or metastasis associated with expression of an oncogene (such as, for example, ABL1, ABL2, ASCL1, AKT1, AKT2, ALK, APC, AR, ARID1A, ARID2, ATM, BCLAF1, BRAF, BRCA1, BRCA2, CCND3, CTNNB1, CREBBP, DNMT3A, EGFR, EP300, ERBB2, ERBB3, ESR1, EZH2, FAT1, FAT3, FAT4, FBXO11, FBXW7, FGFR1, FLT3, FOXA1, GNA11, GNAQ, GNAS, GTF2I, H3F3A, HER2/NEU, HRAS, IDH1, IDH2, JAK2, KCNJ4, KDM6A, KIT, KMT2C, KMT2D, KRAS, LRP1B, MET, MTOR, MYC, MYCN, NF1, NOTCH1, NRAS, NSD1, NSD2, NTRK3, OBSCN, PCBP1, PIK3CA, PIK3R1, PIGR, PLCG1, PTCH1, PTEN, PTPN6, PTPN11, PTPN13, RAC1, RB1, RET, ROS1, RHOA, RYR2, SET2D, SF3B1, SMAD2, SMAD4, SMARCA4, SRC, SRSF2, TP53, TRRAP, TTN, U2AF1L4, VHL, and/or CDKN2A), gene overexpression, or a gene fusion (such as, for example, ALK fusions including, but not limited to EML4-ALK, KIF5B-ALK, HIP1-ALK, KLC1-ALK, DCTN1-ALK, PRKAR1A-ALK, STRN-ALK, CLTC-ALK, MPRIP-ALK, NPM1-ALK, TNS1-ALK, ACTG2-ALK, IGFBP5-ALK, SEC31A-ALK, TPM3-ALK, AITC-ALK, TPM4-ALK; ROS1 fusions including, but not limited to, CD74 ROS1, SLC34A2-ROS1, SDC4-ROS1, EZR-ROS1, LR1G3-ROS1, SDC4-ROS1, TPM3-ROS1, LIMA1-ROS1, MSN-ROS1, WNK1-ROS1, RBPMS-ROS1; or RET fusions including, but not limited to, KIF5B-RET, CCDC6-RET, and TRIM33-RET) in a subject comprising administering to the subject the antigen binding molecule of any preceding aspect (including, but not limited to a dimeric IgA antigen binding molecule that targets KRAS^{G12D}, KRAS^{G12C}, KRAS^{G12V} AKT1^{E17K}-, AKT2^{D324G}, AKT2^{D324H}, IDH1^{R132H}, IDH1^{R132C}, IDH1^{R132L}, ATM^{R337C}, ATM^{R337H} PI3K^{E542K}, PI3K^{E545K}, PI3K^{H1047R}, BRAF^{V600G}, BRAF^{V600E}, SRC^{D407H}, p53^{R273C}, and/or p53^{R273H}). In some aspects, the dimeric antigen molecule can be administered in one or more viral vectors and self-assemble inside the cancerous cell. For example, disclosed herein are methods of treating, inhibiting, decreasing, reducing, ameliorating, and/or preventing a cancer and/or metastasis associated with expression of an oncogene (such as, for example, AKT1 (including, but not limited to AKT1^{E17K}), AKT2 (including, but not limited to AKT2^{D324G} and/or AKT2^{D324H}), AR, ATM (including, but not limited to ATM^{R337C} and/or ATM^{R337H}), BRAF (including, but not limited to BRAF^{V600G} and/or BRAF^{V600E}), ERBB2, ERBB3, ESR1, FOXA1, GNA11, GNAQ, GNAS, HRAS, IDH1 (including, but not limited to IDH1^{R132H}, IDH1^{R132C}, and/or IDH1^{R132L}), IDH2, KCNJ4, KIT, KRAS (including, but not limited to KRAS^{G12D}, KRAS^{G12C}, and/or KRAS^{G12V}), MTOR, MYC, MYCN, NRAS, PI3K (including, but not limited to PI3K^{E542K}, PI3K^{E545K}, and/or PI3K^{H1047R}), PIK3CA, PIGR, PTPN13, p53 (including, but not limited to p53^{R273C} and/or p53^{R273H}), RAC1, SF3B1, SRC (including, but not limited to SRC^{D407H}), TP53, TRRAP, U2AF1L4, and CDKN2A), gene overexpression, or a gene fusion (such as, for example, ALK fusions including, but not limited to EML4-ALK, KIF5B-ALK, HIP1-ALK, KLC1-ALK, DCTN1-ALK, PRKAR1A-ALK, STRN-ALK, CLTC-ALK, MPRIP-ALK, NPM1-ALK, TNS1-ALK, ACTG2-ALK, IGFBP5-ALK, SEC31A-ALK, TPM3-ALK, AITC-ALK, TPM4-ALK; ROS1 fusions including, but not limited to, CD74 ROS1, SLC34A2-ROS1, SDC4-ROS1, EZR-ROS1, LR1G3-ROS1, SDC4-ROS1, TPM3-ROS1, LIMA1-ROS1, MSN-ROS1, WNK1-ROS1, RBPMS-ROS1; or RET fusions including, but not limited to, KIF5B-RET, CCDC6-RET, and TRIM33-RET) in a subject comprising administering to the subject 3 viral vectors (such as, for example, pBMN, adeno-associated virus (AAV), Adenovirus, Herpes virus, Vaccinia virus, Polio virus, AIDS virus (HIV), Sindbis virus, and Murine Maloney Leukemia virus) each comprising different antibody constructs; wherein a first viral construct encodes an antibody J chain; a second viral construct encodes an antibody VH chain, and a third viral construct encodes an antibody VL chain; wherein expression of the VH, VL, and J chains causes self-assembly of a dimeric antigen binding molecule; wherein the dimeric antigen binding molecule binds to the oncogene in the cytosol; and wherein the antigen binding molecule is secreted out of the cell while still captured to the bound the oncogene.

In one aspect, disclosed herein are methods of making a dimeric antigen binding molecule comprising transfecting a cell with 3 viral vectors (such as, for example, pBMN, adeno-associated virus (AAV), Adenovirus, Herpes virus, Vaccinia virus, Polio virus, AIDS virus (HIV), Sindbis virus, and Murine Maloney Leukemia virus) each comprising different antibody constructs; wherein a first viral construct encodes an antibody J chain; a second viral construct encodes an antibody VH chain, and a third viral construct encodes an antibody VL chain; wherein expression of the VH, VL, and J chains causes formation of a dimeric antigen binding molecule.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the generation of a dimeric IgA for IC targeting of mutant KRAS^{G12D}

FIG. 2 shows that the dimeric IgA specifically recognizes KRAS^{G12D}

FIG. 3 shows a cartoon of an experimental design to show that anti-KRAS^{G12D} IgA can specially target G12D-mutated KRAS in intracellular vesicles.

FIG. 4 shows that mutation-specific IgA specifically recognizes intracytoplasmic KRAS^{G12D}

FIG. 5 shows that mutation-specific IgA delays tumor cell progression in a mutation-specific manner.

FIG. 6 shows KRAS^{G12D}-specific IgA1, but NOT KRAS^{G12D}-specific IgG4 (identical VH/VL sequences), abrogate the growth of OVCAR3 cells transduced with KRAS^{G12D}

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular dimeric antigen binding molecules is disclosed and discussed and a number of modifications that can be made to a number of molecules including the dimeric antigen binding molecules are discussed, specifically contemplated is each and every combination and permutation of dimeric antigen binding molecules and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are dimeric IgA, dimeric IgG4, or pentameric IgM antigen binding molecule; wherein the antigen binding molecule specifically binds ABL1, ABL2, ASCL1, AKT1, AKT2, ALK, APC, AR, ARID1A, ARID2, ATM, BCLAF1, BRAF, BRCA1, BRCA2, CCND3, CTNNB1, CREBBP, DNMT3A, EGFR, EP300, ERBB2, ERBB3, ESR1, EZH2, FAT1, FAT3, FAT4, FBXO11, FBXW7, FGFR1, FLT3, FOXA1, GNA11, GNAQ, GNAS, GTF2I, H3F3A, HER2/NEU, HRAS, IDH1, IDH2, JAK2, KCNJ4, KDM6A, KIT, KMT2C, KMT2D, KRAS, LRP1B, MET, MTOR, MYC, MYCN, NF1, NOTCH1, NRAS, NSD1, NSD2, NTRK3, OBSCN, PCBP1, PIK3CA, PIK3R1, PIGR, PLCG1, PTCH1, PTEN, PTPN6, PTPN11, PTPN13, RAC1, RB1, RET, ROS1, RHOA, RYR2, SET2D, SF3B1, SMAD2, SMAD4, SMARCA4, SRC, SRSF2, TP53, TRRAP, TTN, U2AF1L4, VHL, or CDKN2A, gene overexpression, or a gene fusion (such as, for example, ALK fusions including, but not limited to EML4-ALK, KIF5B-ALK, HIP1-ALK, KLC1-ALK, DCTN1-ALK, PRKAR1A-ALK, STRN-ALK, CLTC-ALK, MPRIP-ALK, NPM1-ALK, TNS1-ALK, ACTG2-ALK, IGFBP5-ALK, SEC31A-ALK, TPM3-ALK, AITC-ALK, TPM4-ALK; ROS1 fusions including, but not limited to, CD74 ROS1, SLC34A2-ROS1, SDC4-ROS1, EZR-ROS1, LR1G3-ROS1, SDC4-ROS1, TPM3-ROS1, LIMA1-ROS1, MSN-ROS1, WNK1-ROS1, RBPMS-ROS1; or RET fusions including, but not limited to, KIF5B-RET, CCDC6-RET, and TRIM33-RET); and wherein antigen binding molecule comprising two immunoglobulin monomers comprising a heavy chain, a light chain, and a constant region; wherein the constant regions of the monomers are joined by a J chain. For example, disclosed herein are dimeric or pentameric anti-KRAS^{G12D}-specific, anti-KRAS^{G12C}-specific, anti-KRAS^{G12V}-specific, anti-AKT1^{E17K}-specific, anti-AKT2^{D324G}-specific, anti-AKT2^{D324H}-specific, anti-IDH1^{R132H}-specific, anti-IDH1^{R132C}-specific, anti-IDH1^{R132L}-specific, anti-ATM^{R337C}-specific, anti-ATM^{R337H}-specific, anti-PI3K^{E542K}-specific, anti-PI3K^{E545K}-specific, anti-PI3K^{H1047R}-specific, anti-BRAF^{V600G}-specific, anti-BRAF^{V600E}-specific, anti-SRC^{D407H}-specfic, anti-p53^{R273C}-specific, and/or anti-p53^{R273H}-specific antigen binding molecules comprising two immunoglobulin monomers (such as, for example two IgA or two IgG4) or 5 immunoglobulin monomer (such as, for example, five IgM monomers) comprising a heavy chain, a light chain, and a constant region; wherein the constant regions of the monomers are joined by a J chain. Examples, of antigen binding molecule monomers that can be joined by a J chain and used in the disclosed dimeric antigen binding molecules can be found throughout the art and include, but are not limited to PCT Application No. PCT/IS2021/029517, PCT Application NO. PCT/EP2021/054614, which are incorporated herein by reference for their teachings of anti-KRAS-specific antibodies and the complementarity determining regions for said antibodies. Other examples of antigen binding molecule monomers that can be joined by a J chain and used in the disclosed dimeric antigen binding molecules, include anti-AKT1^{E17K} as available from Thermofisher (Product No. MA5-33069) and from Abcam (Product No. ab238304), anti-IDH1^{R132H} available from Thermofisher (Product No. CF190113) or as disclosed herein in SEQ ID NOs: 15-22, Anti-PI3K^{H1047R} as available from antibodies-online.com (Product No. ABIN6950889), and anti-BRAF^{V600E} as available from Thermofisher (Product No. MA5-24661) or as disclosed herein in SEQ ID NOs: 23-30, each of which is incorporated herein by reference.

For example, the disclosed antigen binding molecule can be the anti-KRAS^{G12D}. Thus, also disclosed herein are dimeric or pentameric anti-KRAS^{G12D}-specific antigen binding molecules (such as, for example, dimeric IgA, dimeric IgG4, or pentameric IgM) wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8; SEQ ID NO: 43, SEQ ID NO: 44, and SEQ ID NO: 45; or SEQ ID NO: 46, SEQ ID NO: 47, and SEQ ID NO: 48, including, but not limited to SEQ ID NO: 5 or SEQ ID NO: 49. In some aspects, the heavy chain variable domain of the dimeric anti-KRAS^{G12D}-specific antigen binding molecules comprises SEQ ID NO: 1, SEQ ID NO: 32, and SEQ ID NO: 34.

In some aspects, disclosed herein are dimeric anti-KRAS^{G12D}-specific antigen binding molecules, wherein the constant region in frame with the heavy chain variable region comprises the sequence as set forth in SEQ ID NO: 2.

Also disclosed herein are dimeric or pentameric anti-KRAS^{G12D}-specific antigen binding molecules (such as, for example, dimeric IgA, dimeric IgG4, or pentameric IgM), wherein the light chain variable domain comprises a complementarity determining region (CDR) 1 (CDR1), CDR2, and CDR3 as set forth in SEQ ID NO: 10, SEQ ID NO: 11, and SEQ ID NO: 12 or SEQ ID NO: 50, SEQ ID NO: 51, and SEQ ID NO: 52, including, but not limited to SEQ ID NO: 9 or SEQ ID NO: 53. In some aspects, the light chain variable domain of the dimeric anti-KRAS^{G12D}-specific antigen binding molecules comprises SEQ ID NO: 3 and SEQ ID NO: 36.

In some aspects, disclosed herein are dimeric anti-KRAS^{G12D}-specific antigen binding molecules, wherein the constant region in frame with the light chain variable region comprises the sequence as set forth in SEQ ID NO: 4.

Also disclosed herein are dimeric or pentameric anti-IDH1^{R132H}-specific antigen binding molecule (such as, for example, dimeric IgA, dimeric IgG4, or pentameric IgM), wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8 or SEQ ID NO: 16, SEQ ID NO: 17, and SEQ ID NO: 18. In some aspects, the variable heavy chain comprises the amino acid sequence as set forth in SEQ ID NO: 15, SEQ ID NO: 38, SEQ ID NO: 40, or SEQ ID NO: 54.

In some aspects, disclosed herein are dimeric or pentameric anti-IDH1^{R132H}-specific antigen binding molecule (such as, for example, dimeric IgA, dimeric IgG4, or pentameric IgM), wherein the light chain variable domain comprises a complementarity determining region (CDR) 1 (CDR1), CDR2, and CDR3 as set forth in SEQ ID NO: 10, SEQ ID NO: 11, and SEQ ID NO: 12 or SEQ ID NO: 20, SEQ ID NO: 21, and SEQ ID NO: 22. In some aspects, the variable light chain comprises the amino acid sequence as set forth in SEQ ID NO: 19, SEQ ID NO: 42, or SEQ ID NO: 55.

### 1. Antibodies

### (1) Antibodies Generally

In some aspects, the disclosed antigen binding molecules are antibodies. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with an oncogene such that the oncogene is bound in the cytosol of a cell and secreted outside the cell while being bound to said oncogene. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, VHH, and the like, including hybrid fragments. Thus, fragments of the antibodies that retain the ability to bind their specific antigens are provided. For example, fragments of antibodies which maintain oncogene (such as KRAS^{G12D}) binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Homology/Identity

It is understood that one way to define any known variants and derivatives or those that might arise, of the disclosed genes and proteins herein is through defining the variants and derivatives in terms of homology to specific known sequences. For example, SEQ ID NO: 13 sets forth a particular sequence of a variable heavy chain of an anti-KRAS^{G12D} antigen binding molecule and SEQID NO: 5 sets forth a particular sequence of the protein encoded by SEQ ID NO: 13. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as dimeric antigen binding molecules into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus (HIV), neuronal trophic virus, Sindbis and other RNA viruses, as well as pBMN. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

In one aspect, disclosed herein are methods of making a dimeric antigen binding molecule comprising transfecting a cell with 3 viral vectors (such as, for example, pBMN, adeno-associated virus (AAV), Adenovirus, Herpes virus, Vaccinia virus, Polio virus, AIDS virus (HIV), Sindbis virus, and Murine Maloney Leukemia virus) each comprising different antibody constructs; wherein a first viral construct encodes an antibody J chain; a second viral construct encodes an antibody VH chain, and a third viral construct encodes an antibody VL chain; wherein expression of the VH, VL, and J chains causes formation of a dimeric antigen binding molecule.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed dimeric antigen binding molecules or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### 4. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHODS OF USING THE COMPOSITIONS

### 1. Method of Treating Cancer

The disclosed compositions can be used to treat any disease where uncontrolled cellular proliferation occurs such as cancers. Also disclosed herein are methods of treating, inhibiting, decreasing, reducing, ameliorating, and/or preventing a cancer and/or metastasis associated with expression of an oncogene (such as, for example, ABL1, ABL2, ASCL1, AKT1, AKT2, ALK, APC, AR, ARID1A, ARID2, ATM, BCLAF1, BRAF, BRCA1, BRCA2, CCND3, CTNNB1, CREBBP, DNMT3A, EGFR, EP300, ERBB2, ERBB3, ESR1, EZH2, FAT1, FAT3, FAT4, FBXO11, FBXW7, FGFR1, FLT3, FOXA1, GNA11, GNAQ, GNAS, GTF2I, H3F3A, HER2/NEU, HRAS, IDH1, IDH2, JAK2, KCNJ4, KDM6A, KIT, KMT2C, KMT2D, KRAS, LRP1B, MET, MTOR, MYC, MYCN, NF1, NOTCH1, NRAS, NSD1, NSD2, NTRK3, OBSCN, PCBP1, PIK3CA, PIK3R1, PIGR, PLCG1, PTCH1, PTEN, PTPN6, PTPN11, PTPN13, RAC1, RB1, RET, ROS1, RHOA, RYR2, SET2D, SF3B1, SMAD2, SMAD4, SMARCA4, SRC, SRSF2, TP53, TRRAP, TTN, U2AF1L4, VHL, and CDKN2A), gene overexpression, or a gene fusion ((such as, for example, ALK fusions including, but not limited to EML4-ALK, KIF5B-ALK, HIP1-ALK, KLC1-ALK, DCTN1-ALK, PRKAR1A-ALK, STRN-ALK, CLTC-ALK, MPRIP-ALK, NPM1-ALK, TNS1-ALK, ACTG2-ALK, IGFBP5-ALK, SEC31A-ALK, TPM3-ALK, AITC-ALK, TPM4-ALK; ROS1 fusions including, but not limited to, CD74 ROS1, SLC34A2-ROS1, SDC4-ROS1, EZR-ROS1, LR1G3-ROS1, SDC4-ROS1, TPM3-ROS1, LIMA1-ROS1, MSN-ROS1, WNK1-ROS1, RBPMS-ROS1; or RET fusions including, but not limited to, KIF5B-RET, CCDC6-RET, and TRIM33-RET) in a subject comprising administering to the subject the any of the dimeric or pentameric antigen binding molecules (such as, for example, dimeric IgA, dimeric IgG4, or pentameric IgM) disclosed herein (including, but not limited to a dimeric IgA antigen binding molecule that targets KRAS^{G12D}, KRAS^{G12C}, KRAS^{G12V}, AKT1^{E17K}-, AKT2^{D324G}, AKT2^{D324H}, IDH1^{R132H}, IDH1^{R132C}, IDH1^{R132L}, ATM^{R337C}, ATM^{R337H}, PI3K^{E542K}, PI3K^{E545K}, PI3K^{H1047R}, BRAF^{V600G}, BRAF^{V600E}, SRC^{D407H}, p53^{R273C}, and/or p53^{R273H}). For example, in one aspect, disclosed herein are methods of treating, inhibiting, decreasing, reducing, ameliorating, and/or preventing a cancer and/or metastasis associated with expression of KRAS mutation in a subject comprising administering to the subject the any of the dimeric or pentameric antigen binding molecules (such as, for example, dimeric IgA, dimeric IgG4, or pentameric IgM); wherein the disclosed antigen binding molecule comprises an anti-KRAS^{G12D}. Thus, also disclosed herein are methods of treating, inhibiting, decreasing, reducing, ameliorating, and/or preventing a cancer and/or metastasis comprising administering a dimeric or pentameric anti-KRAS^{G12D}-specific antigen binding molecules (such as, for example, dimeric IgA, dimeric IgG4, or pentameric IgM) wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8; SEQ ID NO: 43, SEQ ID NO: 44, and SEQ ID NO: 45; or SEQ ID NO: 46, SEQ ID NO: 47, and SEQ ID NO: 48, (including, but not limited to SEQ ID NO: 5 or SEQ ID NO: 49) or having a heavy chain variable domain of the dimeric anti-KRAS^{G12D}-specific antigen binding molecules comprises SEQ ID NO: 1, SEQ ID NO: 32, and SEQ ID NO: 34. In some aspects, disclosed herein methods of treating a cancer with a dimeric or pentameric anti-KRAS^{G12D}-specific antigen binding molecules, wherein the constant region in frame with the heavy chain variable region comprises the sequence as set forth in SEQ ID NO: 2. Also disclosed herein are methods of treating, inhibiting, decreasing, reducing, ameliorating, and/or preventing a cancer and/or metastasis comprising administering a dimeric or pentameric anti-KRAS^{G12D}-specific antigen binding molecules (such as, for example, dimeric IgA, dimeric IgG4, or pentameric IgM), wherein the light chain variable domain comprises a complementarity determining region (CDR) 1 (CDR1), CDR2, and CDR3 as set forth in SEQ ID NO: 10, SEQ ID NO: 11, and SEQ ID NO: 12 or SEQ ID NO: 50, SEQ ID NO: 51, and SEQ ID NO: 52 (including, but not limited to SEQ ID NO: 9 or SEQ ID NO: 53). In some aspects, the light chain variable domain of the dimeric anti-KRAS^{G12D}-specific antigen binding molecules comprises SEQ ID NO: 3 and SEQ ID NO: 36. In some aspects, the dimeric or pentameric anti-KRAS^{G12D}-specific antigen binding molecules used in the disclosed methods comprise a light chain variable region that comprises the sequence as set forth in SEQ ID NO: 4.

Also disclosed herein are disclosed herein are methods of treating, inhibiting, decreasing, reducing, ameliorating, and/or preventing a cancer and/or metastasis associated with expression of a mutated IDH1 comprising administering to the subject a dimeric or pentameric anti-IDH1^{R132H}-specific antigen binding molecule (such as, for example, dimeric IgA, dimeric IgG4, or pentameric IgM), wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8 or SEQ ID NO: 16, SEQ ID NO: 17, and SEQ ID NO: 18. In some aspects, the variable heavy chain comprises the amino acid sequence as set forth in SEQ ID NO: 15, SEQ ID NO: 38, SEQ ID NO: 40, or SEQ ID NO: 54.

In some aspects, disclosed herein are disclosed herein are methods of treating, inhibiting, decreasing, reducing, ameliorating, and/or preventing a cancer and/or metastasis associated with expression of a mutated IDH1 comprising administering to the subject a dimeric or pentameric anti-IDH1^{R132H}-specific antigen binding molecule (such as, for example, dimeric IgA, dimeric IgG4, or pentameric IgM), wherein the light chain variable domain comprises a complementarity determining region (CDR) 1 (CDR1), CDR2, and CDR3 as set forth in SEQ ID NO: 10, SEQ ID NO: 11, and SEQ ID NO: 12 or SEQ ID NO: 20, SEQ ID NO: 21, and SEQ ID NO: 22. In some aspects, the variable light chain comprises the amino acid sequence as set forth in SEQ ID NO: 19, SEQ ID NO: 42, or SEQ ID NO: 55.

In some aspects, the dimeric or pentameric antigen molecule can be administered in one or more viral vectors and self-assemble inside the cancerous cell. For example, disclosed herein are methods of treating, inhibiting, decreasing, reducing, ameliorating, and/or preventing a cancer and/or metastasis associated with expression of an oncogene (such as, for example, AKT1 (including, but not limited to AKT1^{E17K}), AKT2 (including, but not limited to AKT2^{D324G} and/or AKT2^{D324H}), AR, ATM (including, but not limited to ATM^{R337C} and/or ATM^{R337H}), BRAF (including, but not limited to BRAF^{V600G} and/or BRAF^{V600E}), ERBB2, ERBB3, ESR1, FOXA1, GNA11, GNAQ, GNAS, HRAS, IDH1 (including, but not limited to IDH1^{R132H}, IDH1^{R132C}, and/or IDH1^{R132L}), IDH2, KCNJ4, KIT, KRAS (including, but not limited to KRAS^{G12D}, KRAS^{G12C}, and/or KRAS^{G12V}), MTOR, MYC, MYCN, NRAS, PI3K (including, but not limited to PI3K^{E542K}, PI3K^{E545K}, and/or PI3K^{H1047R}), PIK3CA, PIGR, PTPN13, p53 (including, but not limited to p53^{R273C} and/or p53^{R273H}), RAC1, SF3B1, SRC (including, but not limited to SRC^{D407H}), TP53, TRRAP, U2AF1L4, and CDKN2A) in a subject comprising administering to the subject 3 viral vectors (such as, for example, pBMN, adeno-associated virus (AAV), Adenovirus, Herpes virus, Vaccinia virus, Polio virus, AIDS virus (HIV), Sindbis virus, and Murine Maloney Leukemia virus) each comprising different antibody constructs; wherein a first viral construct encodes an antibody J chain; a second viral construct encodes an antibody VH chain, and a third viral construct encodes an antibody VL chain; wherein expression of the VH, VL, and J chains causes self-assembly of a dimeric antigen binding molecule; wherein the dimeric antigen binding molecule binds to the oncogene in the cytosol; and wherein the antigen binding molecule is secreted out of the cell while still captured to the bound the oncogene.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

The disclosed treatments methods can also include the administration any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Treatment methods can include or further include checkpoint inhibitors include, but are not limited to antibodies that block PD-1 (Pembrolizumab, Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

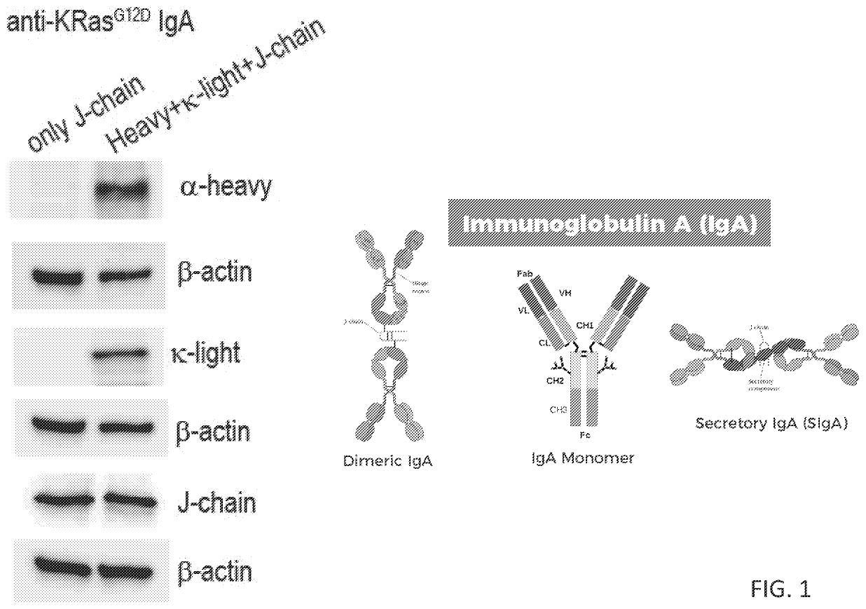

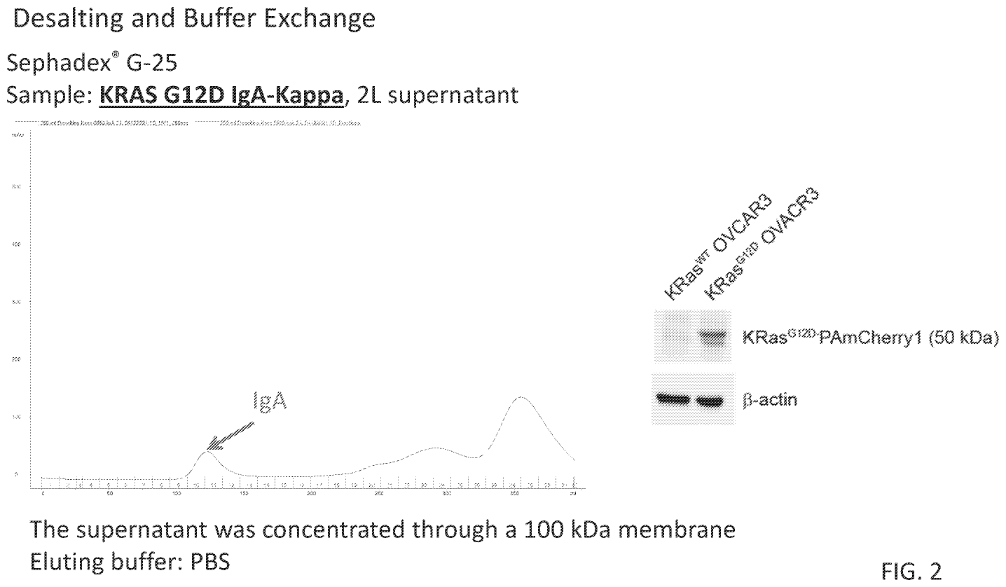

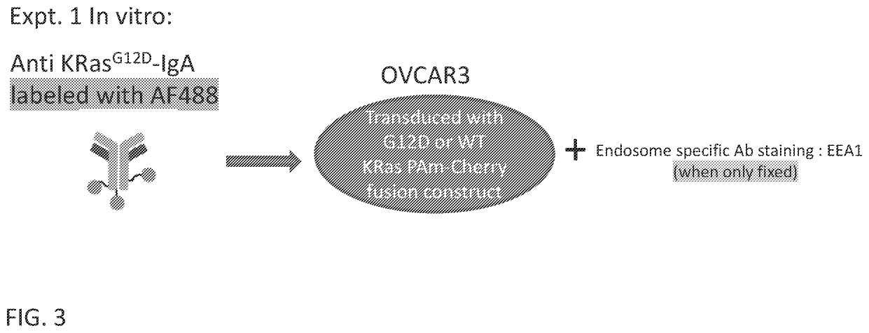

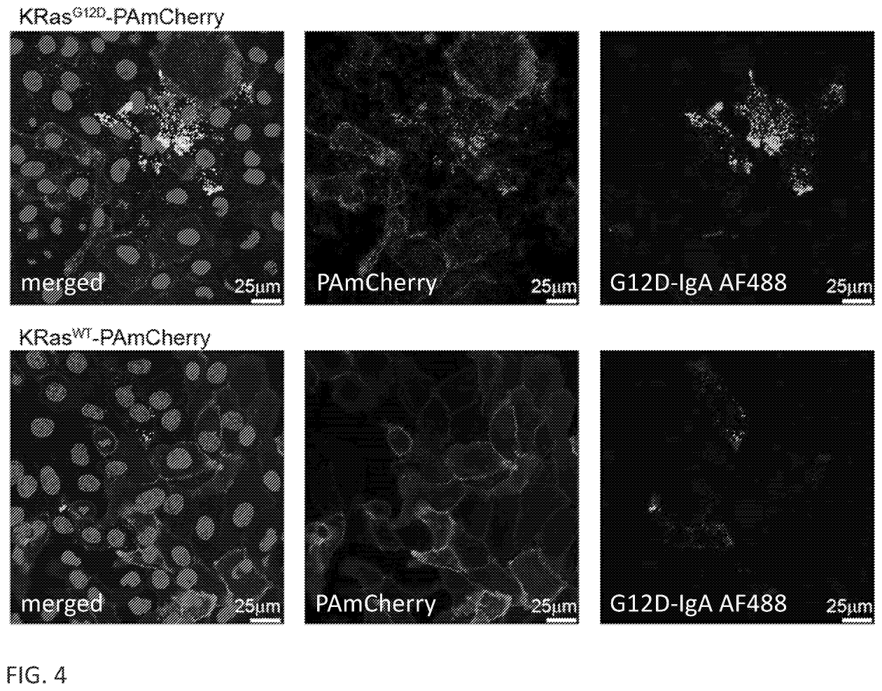

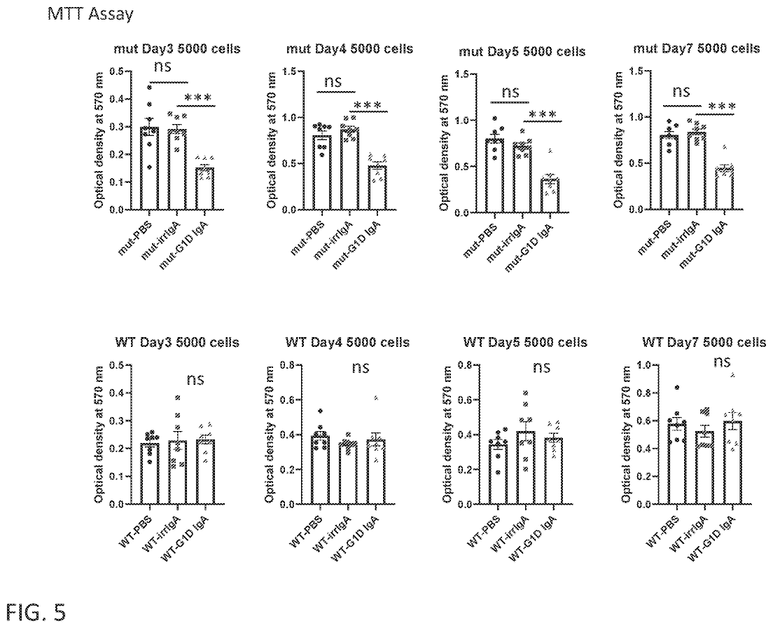

We generated a dimeric IgA for intracytoplasmic targeting of mutant KRAS^{G12D}(FIG. 1). Next, to determine if the dimeric IgA specifically recognizes KRAS^{G12D}, the antibody was concentrated through SEPHADEX® G25 and then western blots run. It was observed that the antibody did specifically recognize KRAS^{G12D} as the no binding was observed with wild-type KRAS (KRAS^{WT}) (FIG. 2). To determine if anti-KRAS^{G12D} IgA can specially target G12D-mutated KRAS in intracellular vesicles, an experiment was designed (FIG. 3) where OVCAR3 cells are transduced with G12D or WT KRAS Pam-cherry fusion construct. The cells were then contacted with anti-KRASg12D IgA labeled with AF488 and viewed using microscopy. Looking at the immunofluorescence staining in FIG. 4, a strong interaction (Co-localization) of G12D IgA Ab was observed, specifically with mutated KRas intracellularly in live condition. Additionally, it was observed that mutation specific IgA Ab treatment translocated mutated Kras (but not) from cell membrane into the cytosol (FIG. 4). To test the ability of G12D specific IgA to inhibit KRASG12D, an MTT assay was conducted. G12D specific IgA, but not non-antigen specific IgA inhibit in vitro KRas^{G12D} expressing OVCAR3 cells (FIG. 5).

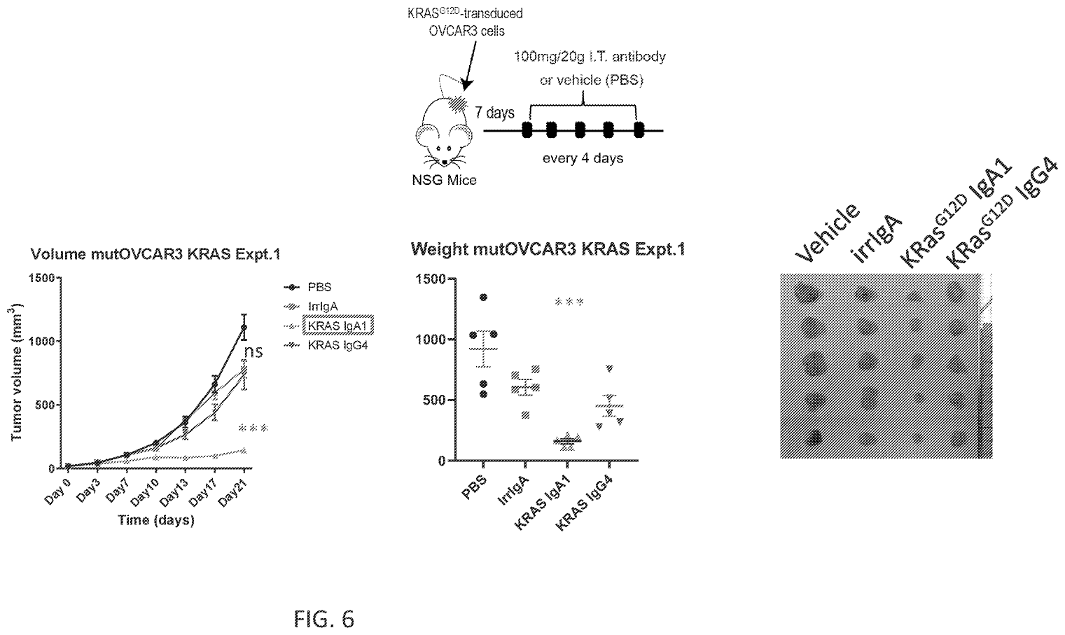

Next we wanted to test the ability to the dimeric antibodies to abrogate tumor growth. We transduced KRAS^{G12D} OVCAR cells into NSG mice. After 7 days and every 4 days thereafter, mice were administered PBS, an irrelevant IgA antibody, a dimeric anti-KRAS^{G12D}-specific IgA antibody, or a dimeric anti-KRAS^{G12D}-specific IgG4 antibody. Tumor volume and weight were measured every 3-4 days after antibody administration for up to 21 days. As shown in FIG. 6, we observed that mice receiving anti-KRAS^{G12D}-specific IgA exhibited >10-fold decrease in tumor growth relative to other groups and ultimately completely stopping tumor growth.

### E. SEQUENCES

anti-KRAS^{G12D} antibody 1 IgA variable heavy chain (VH) VDJ amino acid
sequence
SEQ ID NO: 1
MGWSCIILFLVATATGVHSEVQLVESGGGLVQPGGSLRLSCAASGTFSSYAMSWVRQA
PGKGLEWVSTISRSGHSTYYADSVKGRFTISRDNSKNTLYLQMNSLRAEDTAVYYCAK
RFGSIVFDYWGQGTLVTVSS
anti-KRAS^{G12D} antibody1 IgA heavy chin in frame constant region amino
acid sequence
SEQ ID NO: 2
ASPTSPKVFPLSLCSTQPDGNVVIACLVQGFFPQEPLSVTWWSESGQGVTARNFPPSQDAS
GDLYTTSSQLTLPATQCLAGKSVTCHVKHYTNPSQDVTVPCPVPSTPPTPSPSTPPTPSPS
CCHPRLSLHRPALEDLLLGSEANLTCTLTGLRDASGVTFTWTPSSGKSAVQGPPERDLC
GCYSVSSVLPGCAEPWNHGKTFTCTAAYPESKTPLTATLSKSGNTFRPEVHLLPPPSEEL
ALNELVTLTCLARGFSPKDVLVRWLQGSQELPREKYLTWASRQEPSQGTTTFAVTSILR
VAAEDWKKGDTFSCMVGHEALPLAFTQKTIDRLAGKPTHVNVSVVMAEVDGTCY
anti-KRAS^{G12D} antibody1 variable light chain (VL) VDJ amino acid
sequence
SEQ ID NO: 3
MGWSCIILFLVATATGVHSDLVMTQSPATLSLSPGERATLSCKSSQSLFNSRTRKNYLA
WYQQKPGQAPRLLIYWASTRESGIPGRFSGSGSGTDFLTISSLEPEDFAVYYCKQSYYH
MYTFGQGTKVEIK
anti-KRAS^{G12D} antibody1 IgA light chain in frame constant region amino
acid sequence
SEQ ID NO: 4
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
anti-KRAS^{G12D} binding antibody2 variable heavy chain (VH) amino acid
sequence
SEQ ID NO: 5
MGWSWIFPFLLSGTAGVHCQVVKLQQSGPELVKPGASVKISCKASGYAFSSSWMNWV
KQRPGKGLEWIGRIYPGDGDTNYNGKFKGKATLTADKSSSTANMQLSSLTSEDSAVYF
CAREGSYYTYDVRAYVVYGMDYWGQGTTVTVSS
anti-KRAS^{G12D} binding antibody 2 variable heavy chain (VH)
complementarity determining region (CDR) 1 (CDR1) amino acid sequence
SEQ ID NO: 6
GYAFSSSW
anti-KRAS^{G12D} binding antibody2 VH chain CDR2 amino acid sequence
SEQ ID NO: 7
IYPGDGDT
anti-KRAS^{G12D} binding antibody2 VH chain CDR3 amino acid sequence
SEQ ID NO: 8
AREGSYYTYDVRAYVVYGMDY
anti-KRAS^{G12D} binding antibody2 variable light chain (VL) amino acid
sequence
SEQ ID NO: 9
MDSQAQVLMLLLLWVSGTCGDIVITQSPSSLAVSVGEKVTMRCKSSQSLLYGSNQKNY
LAWYQQKPGQSPKLLIYWASTRESGVPDRFTGSGSGTDFTLTISSVKAEDLAVYYCQQC
YRYPLTFGAGTKLELK
anti-KRAS G12D binding antibody2 variable light chain (VL)
complementarity determining region (CDR) 1 (CDR1) amino acid sequence
SEQ ID NO: 10
QSLLYGSNQKNY
anti-KRAS G12D binding antibody2 VL chain CDR2 amino acid sequence
SEQ ID NO: 11
WAS
anti-KRAS G12D binding antibody2 VL chain CDR3 amino acid sequence
SEQ ID NO: 12
QQCYRYPLT
anti-KRAS G12D binding antibody2 variable heavy chain (VH) nucleic
acid sequence
SEQ ID NO: 13
atgggctggagctggatttttccgtttctgctgagcggcaccgcgggcgtgcattgccaggtggtgaaactgcagcagagcggcccggaa
ctggtgaaaccgggcgcgagcgtgaaaattagctgcaaagcgagcggctatgcgtttagcagcagctggatgaactgggtgaaacagcg
cccgggcaaaggcctggaatggattggccgcatttatccgggcgatggcgataccaactataacggcaaatttaaaggcaaagcgaccct
gaccgcggataaaagcagcagcaccgcgaacatgcagctgagcagcctgaccagcgaagatagcgcggtgtatttttgcgcgcgcgaa
ggcagctattatacctatgatgtgcgcgcgtatgtggtgtatggcatggattattggggccagggcaccaccgtgaccgtgagcagc
anti-KRAS G12D binding antibody2 variable light chain (VL) nucleic acid
sequence
SEQ ID NO: 14
Atggatagccaggcgcaggtgctgatgctgctgctgctgtgggtgagcggcacctgcggcgatattgtgatgagccagagcccgagca
gcctggcggtgagcgtgggcgaaaaagtgaccatggctgcaaaagcagccagagcctgctgtatggcagcaaccagaaaaactatgt
ggcgtggtatcagcagaccccgggccagagcccgaaactgctgatttattgggcgagcacccgcgaaagcggcgtgccggatcgcttta
ccggcagcggcagcggcaccgattttaccctgaccattagcagcgtgaaagcggaagatctggcggtgtattattgccagcagtgctatcg
ctatccgctgacctttggcgcgggcaccaaactggaactgaaa
anti-IDH1^{R132H} binding antibody variable heavy (VH) chain amino acid
sequence
SEQ ID NO: 15
Met Gly Trp Ser Cys Ile Ile Leu Phe Leu Val Ala Ala Thr Gly Val His Ser Gln Val Gln Leu
Gln Gln Pro Gly Thr Glu Leu Val Lys Pro Gly Ala Ser Val His Leu Ser Cys Lys Ala Ser Gly
Tyr Thr Phe Thr Thr Trp Met His Trp Val Glys Gln Arg Pro Gly Gln Gly Leu Glu Trp Ile
Gly Asp Ile Asn Pro Ser Gly Glu Thr Tyr Tyr Asn Glu Lys Phe Arg Asn Lys Ala Thr Leu
Thr Val Asp Lys Ser Ser Arg Thr Ala Tyr Met His Leu Ser Ser Leu Ser Ser Glu Asp Ser Ala Val
Tyr Tyr Cys Ala Arg Gly Phe Lys Asp Tyr Trp Gly Gln Gly Thr Thr Leu Thr Val Ser Ser
anti-IDH1^{R132H} binding antibody VH chain CDR1 amino acid sequence
(residues 50-54 of SEQ ID NO: 15)
SEQ ID NO: 16
Thr Thr Tyr Trp
anti-IDH1^{R132H} binding antibody VH chain CDR2 amino acid sequence
(residues 69-85 of SEQ ID NO: 15)
SEQ ID NO: 17
Asp Ile Asn Pro Ser Asn Gly Glu Thr Tyr Tyr Asn Glu Lys Phe Arg Asn
anti-IDH1^{R132H} binding antibody VH chain CDR3 amino acid sequence
(residues 118-122 of SEQ ID NO: 15)
SEQ ID NO: 18
Gly Phe Lys Asp Tyr
anti-IDH1^{R132H} binding antibody variable light (VL) chain amino acid sequence
SEQ ID NO: 19
Met Lys Leu Pro Val Arg Leu Leu Val Leu Met Phe Trp Ile Pro Val Ala Ser Ser Asp Val Leu
Met Thr Gln Thr Pro Leu Ser Leu Pro Val Ser Leu Gly Asp Gln Ala Ser Ile Ser Cys Arg Ser Ser
Gln Thr Ile Leu Tyr Ser Asp Gly Val Thr Tyr Leu Glu Trp Tyr Leu Gln Lys Pro Gly Gln Ser
Pro Lys Leu Leu Ile Tyr Lys Val Ser Ser Arg Phe Ser Gly Val Pro Asp Arg Phe Ser Gly Ser Gly
Ser Gly Thr Asp Phe Thr Leu Lys Ile Ser Arg Val Val Ala Glu Asp Leu Gly Ile Tyr Tyr Cys Phe
Gln Gly Ser His Val Pro Phe Thr Phe Gly Ser Gly Thr Lys Leu Glu Ile Lys
anti-IDH1^{R132H} binding antibody VL chain CDR1 amino acid sequence
(residues 43-58 of SEQ ID NO: 19)
SEQ ID NO: 20
Ser Ser Gln Thr Ile Leu Tyr Ser Asp Gly Val Thr Tyr Leu Glu
anti-IDH1^{R132H} binding antibody VL chain CDR2 amino acid sequence
(residues 74-80 of SEQ ID NO: 19)
SEQ ID NO: 21
Lys Val Ser Ser Arg Phe Ser
anti-IDH1^{R132H} binding antibody VL chain CDR3 amino acid sequence
(residues 113-121 of SEQ ID NO: 19)
SEQ ID NO: 22
Phe Gln Gly Ser His Val Pro Phe Thr
anti-BRAFV600E binding antibody variable heavy (VH) chain amino acid
sequence (broken underline is artificially added SP underlines are complimentarity
determining regions (CDRs))
SEQ ID NO: 23

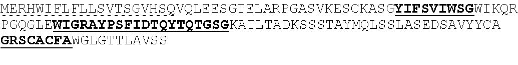

anti-BRAF^{V600E} binding antibody VH chain CDR1 amino acid sequence
SEQ ID NO: 24
YIFSVIWSG
anti-BRAF^{V600E} binding antibody VH chain CDR2 amino acid sequence
SEQ ID NO: 25
WIGRAYPSFIDTQYTQTGSG
anti-BRAFV600E binding antibody VH chain CDR3 amino acid sequence
SEQ ID NO: 26
GRSCACFA
anti-BRAFV600E binding antibody variable light (VL) chain amino acid
sequence (broken underline is artificially added SP underlines are complimentarity
determining regions (CDRs))
SEQ ID NO: 27

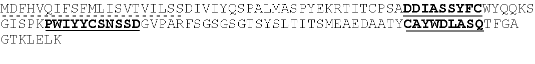

anti-BRAF^{V600E} binding antibody VL chain CDR1 amino acid sequence
SEQ ID NO: 28
DDIASSYFC
anti-BRAF^{V600E} binding antibody VL chain CDR2 amino acid sequence
SEQ ID NO: 29
PWIYYCSNSSD
anti-BRAF^{V600E} binding antibody VL chain CDR3 amino acid sequence
SEQ ID NO: 30
CAYWDLASQ
anti-KRas^{mut} Heavy chain IgA1 nucleic acid
SEQ ID NO: 31
ATGGGCTGGAGCTGCATCATCCTGTTCCTGGTGGCCACCGCCACCGGCGTTCACAGC
GAGGTGCAACTGGTGGAAAGCGGAGGCGGACTCGTTCAGCCCGGCGGATCTCTGCG
GCTGAGCTGCGCCGCCAGCGGCACCTTCAGCAGCTACGCCATGAGCTGGGTCAGAC
AGGCCCCTGGCAAGGGCCTGGAATGGGTGTCCACCATCTCTAGGTCCGGCCACTCT
ACCTACTACGCTGATAGCGTGAAGGGCAGATTTACAATCTCCCGGGACAATAGCAA
GAACACCCTGTACCTGCAGATGAACAGCCTGAGAGCCGAGGACACAGCCGTGTACT
ATTGTGCTAAAAGATTCGGCAGCATCGTGTTCGACTACTGGGGCCAGGGCACACTG
GTGACCGTGTCTAGCGCTAGCCCGACCAGCCCCAAGGTCTTCCCGCTGAGCCTCTGC
AGCACCCAGCCAGATGGGAACGTGGTCATCGCCTGCCTGGTCCAGGGCTTCTTCCCC
CAGGAGCCACTCAGTGTGACCTGGAGCGAAAGCGGACAGGGCGTGACCGCCAGAA
ACTTCCCACCCAGCCAGGATGCCTCCGGGGACCTGTACACCACGAGCAGCCAGCTG
ACCCTGCCGGCCACACAGTGCCTAGCCGGCAAGTCCGTGACATGCCACGTGAAGCA
CTACACGAATCCCAGCCAGGATGTGACTGTGCCCTGCCCAGTTCCCTCAACTCCACC
TACCCCATCTCCCTCAACTCCACCTACCCCATCTCCCTCATGCTGCCACCCCCGACTG
TCACTGCACCGACCGGCCCTTGAGGACCTGCTCTTAGGTTCAGAAGCGAACCTCACG
TGCACACTGACCGGCCTGAGAGATGCCTCAGGTGTCACCTTCACCTGGACGCCCTCA
AGTGGGAAGAGCGCTGTTCAAGGACCACCTGAGCGTGACCTCTGTGGCTGCTACAG
CGTGTCCAGTGTCCTGCCGGGCTGTGCCGAGCCATGGAACCATGGGAAGACCTTCA
CTTGCACTGCTGCCTACCCCGAGTCCAAGACCCCGCTAACCGCCACCCTCTCAAAAT
CCGGAAACACATTCCGGCCCGAGGTCCACCTGCTGCCGCCGCCGTCGGAGGAGCTG
GCCCTGAACGAGCTGGTGACGCTGACGTGCCTGGCACGCGGCTTCAGCCCCAAGGA
CGTGCTGGTTCGCTGGCTGCAGGGGTCACAGGAGCTGCCCCGCGAGAAGTACCTGA
CTTGGGCATCCCGGCAGGAGCCCAGCCAGGGCACCACCACCTTCGCTGTGACCAGC
ATACTGCGCGTGGCAGCCGAGGACTGGAAGAAGGGGGACACCTTCTCCTGCATGGT
GGGCCACGAGGCCCTGCCGCTGGCCTTCACACAGAAGACCATCGACCGCTTGGCGG
GTAAACCCACCCATGTCAATGTGTCTGTTGTCATGGCGGAGGTGGACGGCACCTGCT
ACTGA
anti-KRas^{mut} Heavy chain IgA1 amino acid
SEQ ID NO: 32
MGWSCIILFLVATATGVHSEVQLVESGGGLVQPGGSLRLSCAASGTFSSYAMSWVRQA
PGKGLEWVSTISRSGHSTYYADSVKGRFTISRDNSKNTLYLQMNSLRAEDTAVYYCAK
RFGSIVFDYWGQGTLVTVSSASPTSPKVFPLSLCSTQPDGNVVIACLVQGFFPQEPLSVT
WSESGQGVTARNFPPSQDASGDLYTTSSQLTLPATQCLAGKSVTCHVKHYTNPSQDVT
VPCPVPSTPPTPSPSTPPTPSPSCCHPRLSLHRPALEDLLLGSEANLTCTLTGLRDASGVTF
TWTPSSGKSAVQGPPERDLCGCYSVSSVLPGCAEPWNHGKTFTCTAAYPESKTPLTATL
SKSGNTFRPEVHLLPPPSEELALNELVTLTCLARGFSPKDVLVRWLQGSQELPREKYLT
WASRQEPSQGTTTFAVTSILRVAAEDWKKGDTFSCMVGHEALPLAFTQKTIDRLAGKP
THVNVSVVMAEVDGTCY-
anti-KRas Heavy chain IgG4 nucleic acid
SEQ ID NO: 33
ATGGGCTGGAGCTGCATCATCCTGTTCCTGGTGGCCACCGCCACCGGCGTTCACGC
GAGGTGCAACTGGTGGAAAGCGGAGGCGGACTCGTTCAGCCCGGCGGATCTCTGCG
GCTGAGCTGCGCCGCCAGCGGCACCTTCAGCAGCTACGCCATGAGCTGGGTCAGAC
AGGCCCCTGGCAAGGGCCTGGAATGGGTGTCCACCATCTCTAGGTCCGGCCACTCT
ACCTACTACGCTGATAGCGTGAAGGGCAGATTTACAATCTCCCGGGACAATAGCAA
GAACACCCTGTACCTGCAGATGAACAGCCTGAGAGCCGAGGACACAGCCGTGTACT
ATTGTGCTAAAAGATTCGGCAGCATCGTGTTCGACTACTGGGGCCAGGGCACACTG
GTGACCGTGTCTAGCGCTAGCGCGAGCACCAAAGGCCCGAGCGTGTTTCCGCTGGC
GCCGTGCAGCCGCAGCACCAGCGAAAGCACCGCGGCGCTGGGCTGCCTGGTGAAAG
ATTATTTTCCGGAACCGGTGACCGTGAGCTGGAACAGCGGCGCGCTGACCAGCGGC
GTGCATACCTTTCCGGCGGTGCTGCAGAGCAGCGGCCTGTATAGCCTGAGCAGCGT
GGTGACCGTGCCGAGCAGCAGCCTGGGCACCAAAACCTATACCTGCAACGTGGATC
ATAAACCGAGCAACACCAAAGTGGATAAACGCGTGGAAAGCAAATATGGCCCGCC
GTGCCCGAGCTGCCCGGCGCCGGAATTTCTGGGCGGCCCGAGCGTGTTTCTGTTTCC
GCCGAAACCGAAAGATACCCTGATGATTAGCCGCACCCCGGAAGTGACCTGCGTGG
TGGTGGATGTGAGCCAGGAAGATCCGGAAGTGCAGTTTAACTGGTATGTGGATGGC
GTGGAAGTGCATAACGCGAAAACCAAACCGCGCGAAGAACAGTTTAACAGCACCT
ATCGCGTGGTGAGCGTGCTGACCGTGCTGCATCAGGATTGGCTGAACGGCAAAGAA
TATAAATGCAAAGTGAGCAACAAAGGCCTGCCGAGCAGCATTGAAAAAACCATTAG
CAAAGCGAAAGGCCAGCCGCGCGAACCGCAGGTGTATACCCTGCCGCCGAGCCAG
GAAGAAATGACCAAAAACCAGGTGAGCCTGACCTGCCTGGTGAAAGGCTTTTATCC
GAGCGATATTGCGGTGGAATGGGAAAGCAACGGCCAGCCGGAAAACAACTATAAA
ACCACCCCGCCGGTGCTGGATAGCGATGGCAGCTTTTTTCTGTATAGCCGCCTGACC
GTGGATAAAAGCCGCTGGCAGGAAGGCAACGTGTTTAGCTGCAGCGTGATGCATGA
AGCGCTGCATAACCATTATACCCAGAAAAGCCTGAGCCTGAGCCTGGGCAAATGA
anti-KRas Heavy chain IgG4 amino acid
SEQ ID NO: 34
MGWSCIILFLVATATGVHSEVQLVESGGGLVQPGGSLRLSCAASGTFSSYAMSWVRQA
PGKGLEWVSTISRSGHSTYYADSVKGRFTISRDNSKNTLYLQMNSLRAEDTAVYYCAK
RFGSIVFDYWGQGTLVTVSSASASTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKR
VESKYGPPCPSCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK-
anti-KRas^{mut} Kappa Light chain nucleic acid
SEQ ID NO: 35
ATGGGCTGGAGCTGCATCATCCTGTTCCTGGTGGCCACCGCCACCGGCGTTCACAGC
GATCTGGTGATGACCCAGTCCCCTGCTACACTGAGCCTGTCTCCAGGCGAGCGGGCC
ACCCTGTCCTGCAAGTCCAGCCAGTCTCTCTTCAACAGCAGAACCCGGAAGAACTA
CCTGGCCTGGTATCAGCAGAAGCCTGGCCAGGCCCCTAGACTGCTGATCTACTGGG
CCAGCACCAGAGAGAGCGGCATCCCCGGCAGATTCAGCGGATCTGGCAGCGGAAC
AGACTTTCTGACCATCAGCAGCCTGGAACCCGAGGACTTCGCCGTGTACTATTGTAA
ACAGAGCTACTACCACATGTACACCTTCGGCCAAGGCACAAAGGTGGAAATCAAGC
GAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACA
GAGCAGGACAGCAAGGACAGCACCTACAGCCTCAGCAGCACCCTGACGCTGAGCA
AAGCAGACTACGAGAAACACAAAGTCTACGCCTGCGAAGTCACCCATCAGGGCCTG
AGCTCGCCCGTCACAAAGAGCTTCAACAGGGGAGAGTGTTAG
anti-KRas^{mut} Kappa Light chain amino acid
SEQ ID NO: 36
MGWSCIILFLVATATGVHSDLVMTQSPATLSLSPGERATLSCKSSQSLFNSRTRKNYLA
WYQQKPGQAPRLLIYWASTRESGIPGRFSGSGSGTDFLTISSLEPEDFAVYYCKQSYYH
MYTFGQGTKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSFNR
GEC-
anti-IDH1^{R132H} Heavy chain IgA1 nucleic acid
SEQ ID NO: 37
ATGGGCTGGAGCTGGATTTTTCCGTTTCTGCTGAGCGGCACCGCGGGCGTGCATTGC
CAGGTGGTGAAACTGCAGCAGAGCGGCCCGGAACTGGTGAAACCGGGCGCGAGCG
TGAAAATTAGCTGCAAAGCGAGCGGCTATGCGTTTAGCAGCAGCTGGATGAACTGG
GTGAAACAGCGCCCGGGCAAAGGCCTGGAATGGATTGGCCGCATTTATCCGGGCGA
TGGCGATACCAACTATAACGGCAAATTTAAAGGCAAAGCGACCCTGACCGCGGATA
AAAGCAGCAGCACCGCGAACATGCAGCTGAGCAGCCTGACCAGCGAAGATAGCGC
GGTGTATTTTTGCGCGCGCGAAGGCAGCTATTATACCTATGATGTGCGCGCGTATGT
GGTGTATGGCATGGATTATTGGGGCCAGGGCACCACCGTGACCGTGAGCAGCGCTA
GCCCGACCAGCCCCAAGGTCTTCCCGCTGAGCCTCTGCAGCACCCAGCCAGATGGG
AACGTGGTCATCGCCTGCCTGGTCCAGGGCTTCTTCCCCCAGGAGCCACTCAGTGTG
ACCTGGAGCGAAAGCGGACAGGGCGTGACCGCCAGAAACTTCCCACCCAGCCAGG
ATGCCTCCGGGGACCTGTACACCACGAGCAGCCAGCTGACCCTGCCGGCCACACAG
TGCCTAGCCGGCAAGTCCGTGACATGCCACGTGAAGCACTACACGAATCCCAGCCA
GGATGTGACTGTGCCCTGCCCAGTTCCCTCAACTCCACCTACCCCATCTCCCTCAAC
TCCACCTACCCCATCTCCCTCATGCTGCCACCCCCGACTGTCACTGCACCGACCGGC
CCTTGAGGACCTGCTCTTAGGTTCAGAAGCGAACCTCACGTGCACACTGACCGGCCT
GAGAGATGCCTCAGGTGTCACCTTCACCTGGACGCCCTCAAGTGGGAAGAGCGCTG
TTCAAGGACCACCTGAGCGTGACCTCTGTGGCTGCTACAGCGTGTCCAGTGTCCTGC
CGGGCTGTGCCGAGCCATGGAACCATGGGAAGACCTTCACTTGCACTGCTGCCTAC
CCCGAGTCCAAGACCCCGCTAACCGCCACCCTCTCAAAATCCGGAAACACATTCCG
GCCCGAGGTCCACCTGCTGCCGCCGCCGTCGGAGGAGCTGGCCCTGAACGAGCTGG
TGACGCTGACGTGCCTGGCACGCGGCTTCAGCCCCAAGGACGTGCTGGTTCGCTGG
CTGCAGGGGTCACAGGAGCTGCCCCGCGAGAAGTACCTGACTTGGGCATCCCGGCA
GGAGCCCAGCCAGGGCACCACCACCTTCGCTGTGACCAGCATACTGCGCGTGGCAG
CCGAGGACTGGAAGAAGGGGGACACCTTCTCCTGCATGGTGGGCCACGAGGCCCTG
CCGCTGGCCTTCACACAGAAGACCATCGACCGCTTGGCGGGTAAACCCACCCATGT
CAATGTGTCTGTTGTCATGGCGGAGGTGGACGGCACCTGCTACTGA
anti-IDH1^{R132H} Heavy chain IgA1 amino acid
SEQ ID NO: 38
MGWSWIFPFLLSGTAGVHCQVVKLQQSGPELVKPGASVKISCKASGYAFSSSWMNWV
KQRPGKGLEWIGRIYPGDGDTNYNGKFKGKATLTADKSSSTANMQLSSLTSEDSAVYF
CAREGSYYTYDVRAYVVYGMDYWGQGTTVTVSSASPTSPKVFPLSLCSTQPDGNVVIA
CLVQGFFPQEPLSVTWSESGQGVTARNFPPSQDASGDLYTTSSQLTLPATQCLAGKSVT
CHVKHYTNPSQDVTVPCPVPSTPPTPSPSTPPTPSPSCCHPRLSLHRPALEDLLLGSEANL
TCTLTGLRDASGVTFTWTPSSGKSAVQGPPERDLCGCYSVSSVLPGCAEPWNHGKTFTC
TAAYPESKTPLTATLSKSGNTFRPEVHLLPPPSEELALNELVTLTCLARGFSPKDVLVRW
LQGSQELPREKYLTWASRQEPSQGTTTFAVTSILRVAAEDWKKGDTFSCMVGHEALPL
AFTQKTIDRLAGKPTHVNVSVVMAEVDGTCY-
anti-IDH1^{R132H} Heavy chain IgG4 nucleic acid
SEQ ID NO: 39
ATGGGCTGGAGCTGGATTTTTCCGTTTCTGCTGAGCGGCACCCGCGGGCGTGCATTGC
CAGGTGGTGAAACTGCAGCAGAGCGGCCCGGAACTGGTGAAACCGGGCGCGAGCG
TGAAAATTAGCTGCAAAGCGAGCGGCTATGCGTTTAGCAGCAGCTGGATGAACTGG
GTGAAACAGCGCCCGGGCAAAGGCCTGGAATGGATTGGCCGCATTTATCCGGGCGA
TGGCGATACCAACTATAACGGCAAATTTAAAGGCAAAGCGACCCTGACCGCGGATA
AAAGCAGCAGCACCGCGAACATGCAGCTGAGCAGCCTGACCAGCGAAGATAGCGC
GGTGTATTTTTGCGCGCGCGAAGGCAGCTATTATACCTATGATGTGCGCGCGTATGT
GGTGTATGGCATGGATTATTGGGGCCAGGGCACCACCGTGACCGTGAGCAGCGCTA
GCGCGAGCACCAAAGGCCCGAGCGTGTTTCCGCTGGCGCCGTGCAGCCGCAGCACC
AGCGAAAGCACCGCGGCGCTGGGCTGCCTGGTGAAAGATTATTTTCCGGAACCGGT
GACCGTGAGCTGGAACAGCGGCGCGCTGACCAGCGGCGTGCATACCTTTCCGGCGG
TGCTGCAGAGCAGCGGCCTGTATAGCCTGAGCAGCGTGGTGACCGTGCCGAGCAGC
AGCCTGGGCACCAAAACCTATACCTGCAACGTGGATCATAAACCGAGCAACACCAA
AGTGGATAAACGCGTGGAAAGCAAATATGGCCCGCCGTGCCCGAGCTGCCCGGCGC
CGGAATTTCTGGGCGGCCCGAGCGTGTTTCTGTTTCCGCCGAAACCGAAAGATACCC
TGATGATTAGCCGCACCCCGGAAGTGACCTGCGTGGTGGTGGATGTGAGCCAGGAA
GATCCGGAAGTGCAGTTTAACTGGTATGTGGATGGCGTGGAAGTGCATAACGCGAA
AACCAAACCGCGCGAAGAACAGTTTAACAGCACCTATCGCGTGGTGAGCGTGCTGA
CCGTGCTGCATCAGGATTGGCTGAACGGCAAAGAATATAAATGCAAAGTGAGCAAC
AAAGGCCTGCCGAGCAGCATTGAAAAAACCATTAGCAAAGCGAAAGGCCAGCCGC
GCGAACCGCAGGTGTATACCCTGCCGCCGAGCCAGGAAGAAATGACCAAAAACCA
GGTGAGCCTGACCTGCCTGGTGAAAGGCTTTTATCCGAGCGATATTGCGGTGGAAT
GGGAAAGCAACGGCCAGCCGGAAAACAACTATAAAACCACCCCGCCGGTGCTGGA
TAGCGATGGCAGCTTTTTTCTGTATAGCCGCCTGACCGTGGATAAAAGCCGCTGGCA
GGAAGGCAACGTGTTTAGCTGCAGCGTGATGCATGAAGCGCTGCATAACCATTATA
CCCAGAAAAGCCTGAGCCTGAGCCTGGGCAAATGA
anti-IDH1^{R132H} Heavy chain IgG4 amino acid
SEQ ID NO: 40
MGWSWIFPFLLSGTAGVHCQVVKLQQSGPELVKPGASVKISCKASGYAFSSSWMNWV
KQRPGKGLEWIGRIYPGDGDTNYNGKFKGKATLTADKSSSTANMQLSSLTSEDSAVYF
CAREGSYYTYDVRAYVVYGMDYWGQGTTVTVSSASASTKGPSVFPLAPCSRSTSESTA
ALGCLVKDYFPEPVTVSWNSGALTSGVHTFPAVLQSSGLYSLSSVVTVPSSSLGTKTYT
CNVDHKPSNTKVDKRVESKYGPPCPSCPAPEFLGGPSVFLFPPKPKDTLMISRTPEVTCV
VVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHY
TQKSLSLSLGK-
anti-IDH1^{R132H} Kappa Light Chain nucleic acid
SEQ ID NO: 41
ATGGATAGCCAGGCGCAGGTGCTGATGCTGCTGCTGCTGTGGGTGAGCGGCACCTG
CGGCGATATTGTGATGAGCCAGAGCCCGAGCAGCCTGGCGGTGAGCGTGGGCGAAA
AAGTGACCATGAGCTGCAAAAGCAGCCAGAGCCTGCTGTATGGCAGCAACCAGAA
AAACTATGTGGCGTGGTATCAGCAGACCCCGGGCCAGAGCCCGAAACTGCTGATTT
ATTGGGCGAGCACCCGCGAAAGCGGCGTGCCGGATCGCTTTACCGGCAGCGGCAGC
GGCACCGATTTTACCCTGACCATTAGCAGCGTGAAAGCGGAAGATCTGGCGGTGTA
TTATTGCCAGCAGTGCTATCGCTATCCGCTGACCTTTGGCGCGGGCACCAAACTGGA
ACTGAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGACG
CTGAGCAAAGCAGACTACGAGAAACACAAAGTCTACGCCTGCGAAGTCACCCATCA
GGGCCTGAGCTCGCCCGTCACAAAGAGCTTCAACAGGGGAGAGTGTTAG
anti-IDH1^{R132H} Kappa Light Chain amino acid
SEQ ID NO: 42
MDSQAQVLMLLLLWVSGTCGDIVMSQSPSSLAVSVGEKVTMSCKSSQSLLYGSNQKN
YVAWYQQTPGQSPKLLIYWASTRESGVPDRFTGSGSGTDFTLTISSVKAEDLAVYYCQQ
CYRYPLTFGAGTKLELKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKS
FNRGEC-
anti-KRAS^{mut} Heavy Chain CDR1
SEQ ID NO: 43
SYAMS
anti-KRAS^{mut} Heavy Chain CDR2
SEQ ID NO: 44
TISRSGHSTYYADSVKG
anti-KRAS^{mut} Heavy Chain CDR3
SEQ ID NO: 45
RFGSIVFDY
anti-KRAS^{mut} Heavy Chain CDR1
SEQ ID NO: 46
GTFSSYA
anti-KRAS^{mut} Heavy Chain CDR2
SEQ ID NO: 47
ISRSGHST
anti-KRAS^{mut} Heavy Chain CDR3
SEQ ID NO: 48
AKRFGSIVFDY
anti-KRAS^{G12D} Heavy Chain amino acid sequence
SEQ ID NO: 49

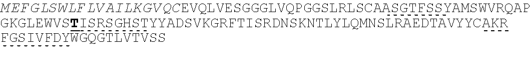

anti-KRAS^{G12D} binding antibody2 variable light chain (VL)
complementarity determining region (CDR) 1 (CDR1) amino acid sequence
SEQ ID NO: 50
QSLLYGSNQKNYL
anti-KRAS^{G12D} binding antibody2 VL chain CDR2 amino acid sequence
SEQ ID NO: 51
WAS
anti-KRAS^{G12D} binding antibody2 VL chain CDR3 amino acid sequence
SEQ ID NO: 52
KQSYYHMYT
Humanized anti-KRAS^{G12D} binding antibody variable light chain amino
acid sequence- CDRs broken underlined
SEQ ID NO: 53

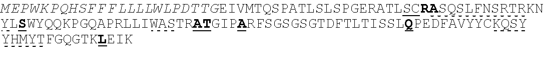

Humanized anti-IDH1^{R132H} variable heavy chain amino acid sequence-
CDRs broken underlined
SEQ ID NO: 54

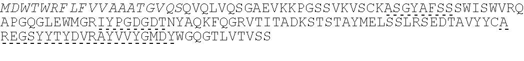

Humanized anti-IDH1^{R132H} variable light chain amino acid sequence-
CDRs broken underlined
SEQ ID NO: 55

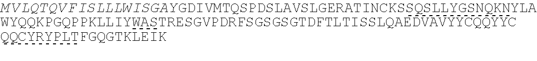

Anti-Myc Ab VH (mouse; human constant IgA1 underlined; CDR regions
broken underline):
SEQ ID NO: 56

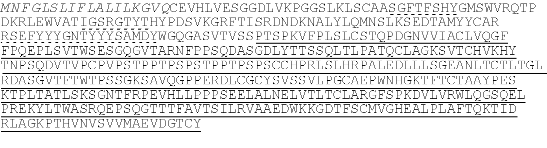

Anti-Myc Ab Humanized VH on IGHV3-21*01/IGHJ4*01:
SEQ ID NO: 57
MELGLRWVFLVAILEGVQCEVQLVESGGGLVKPGGSLRLSCAASGFTFSHYGMNWVRQ
APGKGLEWVSSIGSRGTYTYYADSVKGRFTISRDNAKNSLYLQMNSLRAEDTAVYYCA
RRSEFYYYGNTYYYSAMDYWGQGTLVTVSS
Anti-Myc Ab VL Kappa (mouse; human constant kappa underlined):
SEQ ID NO: 58
MEKDTLLLWVLLLWVPGSTGDIVLTSPASLAVSLGQRATISCRASEESVDNYGFSFMNWF
QQKPGQPPKLLIYAISNRGSGVPARFSGSGSGTDFSLNIHPVEEDDPAMYFCQQTKEVPW
TFGGGTKLEIKASPTSPKVFPLSLCSTQPDGNVVIACLVQGFFPQEPLSVTWSESGQGVT
ARNFPPSQDASGDLYTTSSQLTLPATQCLAGKSVTCHVKHYTNPSQDVTVPCPVPSTPPT
PSPSTPPTPSPSCCHPRLSLHRPALEDLLLGSEANLTCTLTGLRDASGVTFTWTPSSGKSA
VQGPPERDLCGCYSVSSVLPGCAEPWNHGKTFTCTAAYPESKTPLTATLSKSGNTFRPE
VHLLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKPTHVNVSVVMA
EVDGTCY
Anti-Myc Ab Humanized on IGKV3D-11*02/IGKJ4*01:
SEQ ID NO: 59
MEAPAQLLFLLLLWLPDTTGEIVLTQSPATLSLSPGERATLSCRASESVDNYGFSFLSQYQ
QKPGQAPRLLIYAISNRATGIPARFSGSGPGTDFTLTISSLEPEDFAVYYCQQTKEVPWTF
GGGTKVEIK
Anti-ASCL1VH (mouse); CDR regions broken underlined:
SEQ ID NO: 60

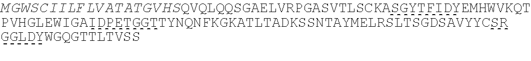

Anti-ASCL1 Humanized on IGHV1-2*06/IGHJ6*01: CDR regions
broken underline
SEQ ID NO: 61

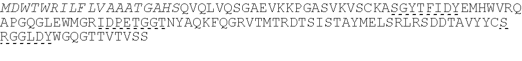

Anti-ASCL1VH CDR1
SEQ ID NO: 62

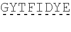

Anti-ASCL1VH CDR2
SEQ ID NO: 63

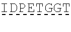

Anti-ASCL1VH CDR3
SEQ ID NO: 64

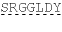

Anti-ASCL1VL Kappa (mouse); CDR regions broken underline:
SEQ ID NO: 65

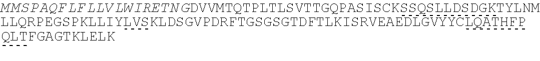

Anti-ASCL1 Humanized on IGV2-29*02/IGKJ2*01; CDR regions
broken underline:
SEQ ID NO: 66

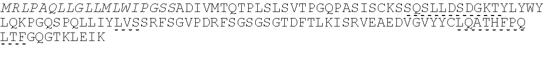

Anti-ASCL1VL CDR1
SEQ ID NO: 67

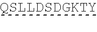

Anti-ASCL1VL CDR2
SEQ ID NO: 68

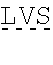

Anti-ASCL1VL CDR3
SEQ ID NO: 69

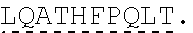

1. A dimeric IgA, dimeric IgG4, or pentameric IgM antigen binding molecule; wherein the antigen binding molecule specifically binds ABL1, ABL2, ASCL1, AKT1, AKT2, ALK, APC, AR, ARID1A, ARID2, ATM, BCLAF1, BRAF, BRCA1, BRCA2, CCND3, CTNNB1, CREBBP, DNMT3A, EGFR, EP300, ERBB2, ERBB3, ESR1, EZH2, FAT1, FAT3, FAT4, FBXO11, FBXW7, FGFR1, FLT3, FOXA1, GNA11, GNAQ, GNAS, GTF2I, H3F3A, HER2/NEU, HRAS, IDH1, IDH2, JAK2, KCNJ4, KDM6A, KIT, KMT2C, KMT2D, KRAS, LRP1B, MET, MTOR, MYC, MYCN, NF1, NOTCH1, NRAS, NSD1, NSD2, NTRK3, OBSCN, PCBP1, PIK3CA, PIK3R1, PIGR, PLCG1, PTCH1, PTEN, PTPN6, PTPN11, PTPN13, RAC1, RB1, RET, ROS1, RHOA, RYR2, SET2D, SF3B1, SMAD2, SMAD4, SMARCA4, SRC, SRSF2, TP53, TRRAP, TTN, U2AF1L4, VHL, or CDKN2A; and wherein antigen binding molecule comprising two immunoglobulin monomers comprising a heavy chain, a light chain, and a constant region; wherein the constant regions of the monomers are joined by a J chain.

2. The dimeric IgA, dimeric IgG4, or pentameric IgM antigen binding molecule of claim 1, wherein the antigen binding molecule is an anti-KRAS^{G12D}-specific antigen binding molecule.

3. The dimeric IgA, dimeric IgG4, or pentameric IgM anti-KRAS^{G12D} antigen binding molecule of claim 2, wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8; SEQ ID NO: 43, SEQ ID NO: 44, and SEQ ID NO: 45; or SEQ ID NO: 46, SEQ ID NO: 47, and SEQ ID NO: 48.

4. The dimeric IgA, dimeric IgG4, or pentameric IgM anti-KRAS^{G12D} antigen binding molecule of claim 2, wherein the variable heavy chain comprises the amino acid sequence as set forth in SEQ ID NO: 1, SEQ ID NO: 5, SEQ ID NO: 32, SEQ ID NO: 34, and SEQ ID NO: 49.

5. The dimeric IgA, dimeric IgG4, or pentameric IgM anti-KRAS^{G12D} antigen binding molecule of claim 2 wherein the constant region in frame with the heavy chain variable region comprises the sequence as set forth in SEQ ID NO: 2.

6. The dimeric IgA, dimeric IgG4, or pentameric IgM anti-KRAS^{G12D} antigen binding molecule of claim 2, wherein the light chain variable domain comprises a CDR1, CDR2, and CDR3 as set forth in SEQ ID NO: 10, SEQ ID NO: 11, and SEQ ID NO: 12 or SEQ ID NO: 50, SEQ ID NO: 51, and SEQ ID NO: 52.

7. The dimeric IgA, dimeric IgG4, or pentameric IgM anti-KRAS^{G12D} antigen binding molecule of claim 2, wherein the variable light chain comprises the amino acid sequence as set forth in SEQ ID NO: 3, SEQ ID NO: 9, SEQ ID NO: 36, and SEQ ID NO: 53.

8. The dimeric IgA, dimeric IgG4, or pentameric IgM anti-KRAS^{G12D} antigen binding molecule of claim 2 wherein the constant region in frame with the heavy chain variable region comprises the sequence as set forth in SEQ ID NO: 4.

9. (canceled)

10. The dimeric IgA, dimeric IgG4, or pentameric IgM antigen binding molecule of claim 1, wherein the antigen binding molecule is an anti-IDH1^{R132H}-specific antigen binding molecule.

11. The dimeric IgA, dimeric IgG4, or pentameric IgM anti-IDH1^{R132H} antigen binding molecule of claim 10, wherein the heavy chain variable domain comprises a complementarity determining region (CDR) 1 (CDR1), CDR2, and CDR3 as set forth in SEQ ID NO: 6, SEQ ID NO: 7, and SEQ ID NO: 8 or SEQ ID NO: 16, SEQ ID NO: 17, and SEQ ID NO: 18.

12. The dimeric IgA, dimeric IgG4, or pentameric IgM anti-IDH1^{R132H} antigen binding molecule of claim 10, wherein the variable heavy chain comprises the amino acid sequence as set forth in SEQ ID NO: 15, SEQ ID NO: 38, SEQ ID NO: 40, or SEQ ID NO: 54.

13. The dimeric IgA, dimeric IgG4, or pentameric IgM antigen binding molecule of claim 10, wherein the variable light chain comprises a CDR1, CDR2, and CDR3 as set forth in SEQ ID NO: 10, SEQ ID NO: 11, and SEQ ID NO: 12 or SEQ ID NO: 20, SEQ ID NO: 21, and SEQ ID NO: 22.

14. The dimeric IgA, dimeric IgG4, or pentameric IgM anti-IDH1^{R132H} antigen binding molecule of claim 10, wherein the variable light chain comprises the amino acid sequence as set forth in SEQ ID NO: 9, SEQ ID NO: 19, SEQ ID NO: 42, or SEQ ID NO: 55.

15. (canceled)

16. The dimeric IgA, dimeric IgG4, or pentameric IgM antigen binding molecule of claim 1, wherein the antigen binding molecule is an anti-ASCL1-specific antigen binding molecule.

17. The dimeric IgA, dimeric IgG4, or pentameric IgM anti-ASCL1 antigen binding molecule of claim 2, wherein the heavy chain variable domain comprises a complementarity determining region (CDR) 1 (CDR1), CDR2, and CDR3 as set forth in SEQ ID NO: 62, SEQ ID NO: 63, and SEQ ID NO: 64.

18. The dimeric IgA, dimeric IgG4, or pentameric IgM anti-ASCL1 antigen binding molecule of claim 2, wherein the variable heavy chain comprises the amino acid sequence as set forth in SEQ ID NO: 60 and SEQ ID NO: 61.

19. The dimeric IgA, dimeric IgG4, or pentameric IgM anti-ASCL1 antigen binding molecule of claim 2, wherein the light chain variable domain comprises a CDR1, CDR2, and CDR3 as set forth in SEQ ID NO: 67, SEQ ID NO: 68, and SEQ ID NO: 69.

20. The dimeric IgA, dimeric IgG4, or pentameric IgM anti-ASCL1 antigen binding molecule of claim 2, wherein the variable light chain comprises the amino acid sequence as set forth in SEQ ID NO: 65 and SEQ ID NO: 66.

21. A method of treating a cancer in a subject comprising administering to the subject the antigen binding molecule of claim 1.

22. A method of treating a cancer associated with an oncogene or fusion in a subject comprising administering to the subject 3 viral vectors each comprising different antibody constructs; wherein a first viral construct encodes an antibody J chain; a second viral construct encodes an antibody VH chain, and a third viral construct encodes an antibody VL chain; wherein expression of the VH, VL, and J chains causes self-assembly of a dimeric antigen binding molecule; wherein the dimeric antigen binding molecule binds to the oncogene or fusion in the cytosol; and wherein the antigen binding molecule is secreted out of the cell while still captured to the bound the oncogene.

23. (canceled)

24. (canceled)

25. The method of treating a cancer in a subject of claim 22, wherein the oncogene is selected from the group consisting of ABL1, ABL2, ASCL1, AKT1, AKT2, ALK, APC, AR, ARID1A, ARID2, ATM, BCLAF1, BRAF, BRCA1, BRCA2, CCND3, CTNNB1, CREBBP, DNMT3A, EGFR, EP300, ERBB2, ERBB3, ESR1, EZH2, FAT1, FAT3, FAT4, FBXO11, FBXW7, FGFR1, FLT3, FOXA1, GNA11, GNAQ, GNAS, GTF2I, H3F3A, HER2/NEU, HRAS, IDH1, IDH2, JAK2, KCNJ4, KDM6A, KIT, KMT2C, KMT2D, KRAS, LRP1B, MET, MTOR, MYC, MYCN, NF1, NOTCH1, NRAS, NSD1, NSD2, NTRK3, OBSCN, PCBP1, PIK3CA, PIK3R1, PIGR, PLCG1, PTCH1, PTEN, PTPN6, PTPN11, PTPN13, RAC1, RB1, RET, ROS1, RHOA, RYR2, SET2D, SF3B1, SMAD2, SMAD4, SMARCA4, SRC, SRSF2, TP53, TRRAP, TTN, U2AF1L4, VHL, or CDKN2A.

26. (canceled)

27. (canceled)

28. The method of treating a cancer in a subject of claim 22, wherein the fusion comprises a fusion selected from the group consisting of EML4-ALK, KIF5B-ALK, HIP1-ALK, KLC1-ALK, DCTN1-ALK, PRKAR1A-ALK, STRN-ALK, CLTC-ALK, MPRIP-ALK, NPM1-ALK, TNS1-ALK, ACTG2-ALK, IGFBP5-ALK, SEC31A-ALK, TPM3-ALK, AITC-ALK, TPM4-ALK, CD74 ROS1, SLC34A2-ROS1, SDC4-ROS1, EZR-ROS1, LR1G3-ROS1, SDC4-ROS1, TPM3-ROS1, LIMA1-ROS1, MSN-ROS1, WNK1-ROS1, RBPMS-ROS1, KIF5B-RET, CCDC6-RET, and TRIM33-RET.
